(19) United States
(12) Patent Application Publication
(10) Pub. No.: US 2011/0202104 A1
(43) Pub. Date: Aug. 18, 2011

(54) METHOD AND SYSTEM FOR AUTOMATICALLY SWITCHING BETWEEN MODES OF AN IMPLANTABLE MEDICAL DEVICE BASED ON AN EXTERNAL MAGNETIC FIELD

(75) Inventor: Neha Butala, Valencia, CA (US)

(73) Assignee: PACESETTER, INC., Sylmar, CA (US)

(21) Appl. No.: 12/707,562

(22) Filed: Feb. 17, 2010

(51) Int. Cl. A61B 5/0428; A61N 1/08

(52) U.S. Cl. 607/30; 600/409

(57) ABSTRACT

An implantable medical device that is configured to be exposed to magnetic fields includes a lead, a detection module, a field measurement sensor, and a control module. The lead includes electrodes that are positioned within a heart to sense cardiac signals of the heart. The detection module monitors the cardiac signals to identify cardiac events based on the cardiac signals. The field measurement sensor measures a magnetic field. The sensor generates a field measurement based on the measured magnetic field. The sensor remains in an unsaturated state when exposed to the magnetic field of at least 0.2 Tesla. The control module identifies a presence of the magnetic field based on the field measurement of the sensor and switches operation of the detection module to an MR safe mode based on the field measurement.

### FIELD OF THE INVENTION

Embodiments of the present invention generally pertain to implantable medical devices and more particularly to methods and systems that switch modes of operation of an implantable medical device based on the presence of an external magnetic field.

### BACKGROUND OF THE INVENTION

An implantable medical device (IMD) is implanted in a patient to monitor, among other things, electrical activity of a heart and to deliver appropriate electrical therapy, as required. Implantable medical devices include pacemakers, cardioverters, defibrillators, implantable cardioverter defibrillators (ICD), and the like. The electrical therapy produced by an IMD may include pacing pulses, cardioverting pulses, and/or defibrillator pulses to reverse arrhythmias (e.g., tachycardias and bradycardias) or to stimulate the contraction of cardiac tissue (e.g., cardiac pacing) to return the heart to its normal sinus rhythm.

Magnetic fields may be produced by magnetic resonance (MR) imaging systems that generate relatively strong magnetic fields. For example, some known commercial MR imaging systems create magnetic fields on the order of 0.5 to 3.0 Tesla. When IMDs are exposed to external magnetic fields such as those of MR imaging systems, the fields may interfere with operation of the IMD. For example, an external magnetic field may generate magnetic forces on the IMD and on leads and electrodes of the IMD. These forces may induce electric charges or potential on the leads and electrodes. The electric charges can cause over- or under-sensing of cardiac signals in the electrodes and leads. For example, the charges may cause the electrodes and leads to convey signals to the IMD that are not cardiac signals but are treated by the IMD as cardiac signals. In another example, the charges may induce sufficient noise in the cardiac signals such that cardiac signals that are representative of a cardiac event go undetected by the IMD.

MR Imaging systems may generate external magnetic fields of have different strengths, such as 0.5 Tesla, 0.7 Tesla, 1.0 Tesla, 1.2 Tesla, 1.5 Tesla, 3 Tesla, etc. Some IMDs may operate safely, while in certain modes, when exposed to lower strength magnetic fields. However, when IMDs are exposed to higher magnetic fields, the IMDs may be unable to reliably operate in a physiologic safe manner in most modes. In order to safely operate in some external magnetic fields, the IMDs may switch modes to an “MR safe mode” or a “magnet mode.”

In order to sense and detect external magnetic fields, some IMDs include Giant Magnetoresistance (GMR) sensors. Known GMR sensors are limited to detecting magnetic fields of relatively small magnitudes. The GMR sensor operates by detecting a large change in an electrical resistance characteristic of the sensor when the sensor is exposed to the magnetic field. The change in electrical resistance is used by IMD to detect proximity of magnetic fields of relatively small magnitudes. In response, the IMD switches to the magnet mode of operation. During the magnet mode of operation, the IMD paces the ventricle at a predetermined fixed rate and does not sense cardiac signals or respond to any detected cardiac events.

Conventional GMR sensors used in IMDs are formed from materials that may become saturated when exposed to relatively small magnetic fields. For example, some known GMR sensors become saturated when exposed to magnetic fields of approximately 15 Gauss. Once saturated, the electrical resistance of a saturated sensor does not change even as the external magnetic field changes. Once the sensor is saturated, further increases in the external magnetic field are not detected by the sensor. MR imaging systems can generate magnetic fields that are significantly larger than 15 Gauss. Therefore, the sensors are unable to detect whether the external magnetic field of an MR imaging system becomes very strong and potentially dangerous for the continued operation of the IMD, even in an MR safe mode or a magnet mode of operation.

These sensors may be unable to reliably sense relatively strong external magnetic fields. As a result, the GMR sensors may be incapable of detecting the presence of an external magnetic field that is generated by some MR imaging systems. Also, the GMR sensors may be unable to differentiate between different magnetic fields. For example, the GMR sensors may be incapable of differentiating between relatively small external magnetic fields, in which the IMD may continue to safely operate, and relatively strong external magnetic fields, in which the IMDs may be unable to safely operate.

Therefore, a need exists for an IMD that is able to sense the exposure of the IMD to external magnetic fields generated by MR imaging systems, detect the magnitude of the external magnetic fields, and/or differentiate between the external magnetic fields of different MR imaging systems, or different external magnetic fields, without being saturated by the magnetic fields.

### BRIEF SUMMARY OF THE INVENTION

In one embodiment, an implantable medical device that is configured to be exposed to magnetic fields includes a lead, a detection module, a field measurement sensor, and a control module. The lead includes electrodes that are positioned within a heart to sense cardiac signals of the heart. The detection module monitors the cardiac signals to identify cardiac events based on the cardiac signals. The field measurement sensor measures a magnetic field. The sensor generates a field measurement based on the measured magnetic field. The sensor remains in an unsaturated state when exposed to the magnetic field of at least 0.2 Tesla. The control module identifies a presence of the magnetic field based on the field measurement of the sensor and switches operation of the detection module to an MR safe mode based on the field measurement.

In another embodiment, a method for switching modes of an implantable medical device based on a magnetic field is provided. The method includes sensing cardiac signals originating from a heart over electrodes positioned within the heart and monitoring the cardiac signals to identify cardiac events. The method further includes measuring the magnetic field to which the implantable medical device is exposed to generate a field measurement using a field measurement sensor. The field measurement sensor remains in an unsaturated state when exposed to a magnetic field of at least 0.2 Tesla. The method also includes recognizing the presence of the magnetic field based on the field measurement of the sensor and switching operation of the implantable medical device to an MR safe mode based on the field measurement.

In another embodiment, another implantable medical device (IMD) is provided. The IMD includes a lead, a detection module, a field measurement sensor, and a control module. The lead includes electrodes that are configured to be positioned within a heart to sense cardiac signals of the heart. The detection module monitors the cardiac signals to identify cardiac events based on the cardiac signals. The field measurement sensor measures a magnetic field to which the implantable medical device is exposed and generates a corresponding field measurement. The sensor includes alternating layers of paramagnetic material and conductive material. The control module identifies a presence of the magnetic field based on the field measurement of the sensor and switches operation of the detection module to an MR safe mode based on the field measurement.

### BRIEF DESCRIPTION OF THE DRAWINGS

The drawings illustrate generally, by way of example, but not by way of limitation, various embodiments discussed in the present document.

FIG. 1 illustrates an implantable medical device (IMD) coupled to a heart in accordance with one embodiment.

FIG. 2 is a schematic illustration of a low permeability sensing resistive element in accordance with one embodiment.

FIG. 3 illustrates a schematic diagram of magnetic dipole moments of electrons in a paramagnetic layer of the resistive element shown in FIG. 2 in accordance with one embodiment.

FIG. 4 illustrates a schematic diagram of the magnetic dipole moments of the paramagnetic layer shown in FIG. 3 when exposed to a first external magnetic field in accordance with one embodiment.

FIG. 5 illustrates a schematic diagram of the magnetic dipole moments of the paramagnetic layer shown in FIG. 4 when exposed to a second external magnetic field in accordance with one embodiment.

FIG. 6 illustrates a schematic diagram of the magnetic dipole moments of the paramagnetic layer shown in FIG. 5 when exposed to a third external magnetic field in accordance with one embodiment.

FIG. 7 is a schematic illustration of the resistive element shown in FIG. 2 exposed to a relatively large external magnetic field in accordance with one embodiment.

FIG. 8 is a circuit diagram of the sensor shown in FIG. 1 in accordance with one embodiment.

FIG. 9 illustrates an example of a relationship between a voltage differential due to detected resistance change by the sensor (shown in FIG. 1) and the strength of an external magnetic field (shown in FIG. 7) in accordance with one embodiment.

FIG. 10 is a flowchart of a method for switching modes of the IMD (shown in FIG. 1) based on a magnetic field in accordance with one embodiment.

FIG. 11 illustrates a block diagram of exemplary internal components of the IMD (shown in FIG. 1) in accordance with one embodiment.

FIG. 12 illustrates a block diagram of example manners in which embodiments of the present invention may be stored, distributed, and installed on a computer-readable medium.

FIG. 13 is a schematic illustration of a low permeability sensing resistive element in accordance with another embodiment.

FIG. 14 is a schematic illustration of a low permeability sensing resistive element in accordance with another embodiment.

FIG. 15 is a schematic illustration of a low permeability sensing resistive element in accordance with another embodiment.

FIG. 16 is a schematic illustration of a field measurement sensor in accordance with another embodiment.

FIG. 17 illustrates several relationships between electrical resistivity of resistive elements shown in FIG. 8 and external magnetic fields in accordance with one embodiment.

FIG. 18 illustrates several other relationships between electrical resistivity of resistive elements shown in FIG. 8 and external magnetic fields in accordance with one embodiment.

### DETAILED DESCRIPTION OF THE INVENTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. The following detailed description is, therefore, not to be taken in a limiting sense, and the scope of the present invention is defined by the appended claims and their equivalents. In this document, the terms “a” or “an” are used, as is common in patent documents, to include one or more than one. In this document, the term “or” is used to refer to a nonexclusive or, unless otherwise indicated.

In accordance with certain embodiments, methods and systems are provided for automatically detecting entry of a patient who has an implantable medical device (IMD) into an MRI room that includes a relatively large magnetic field and automatically switching operation of the IMD to an MR safe mode while the IMD is in the magnetic field. The IMD may detect the strength of the external magnetic field and distinguish between different external magnetic field strengths. Switching operation of the IMD to a safe mode of operation when the IMD is in the magnetic field may prevent the IMD from over- or under-sensing cardiac signals of the patient. Once the patient and the IMD exit the magnetic field, the IMD may switch back to a normal mode of operation. For example, the IMD may return to the operating state used by the IMD prior to entering the MRI room.

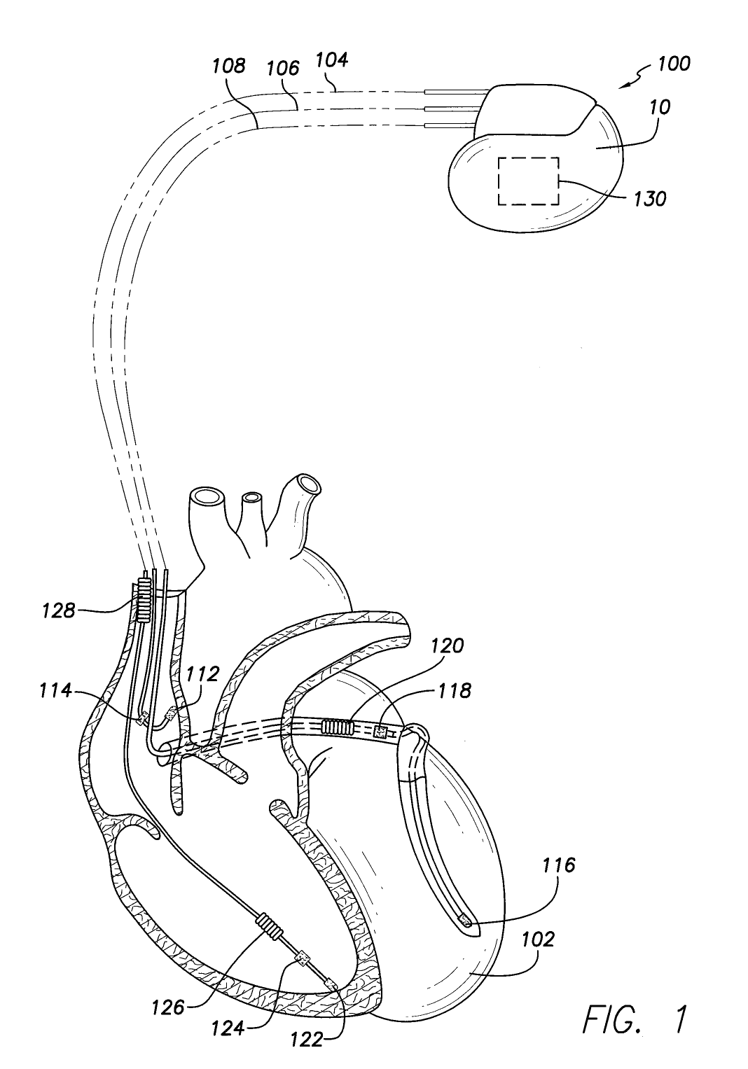

FIG. 1 illustrates an implantable medical device (IMD) 100 coupled to a heart 102 in accordance with one embodiment. The IMD 100 may be a cardiac pacemaker, an ICD, a defibrillator, an ICD coupled with a pacemaker, a cardiac resynchronization therapy (CRT) pacemaker, a cardiac resynchronization therapy defibrillator (CRT-D), and the like. The IMD 100 includes a housing 110 that is joined to several leads 104, 106, 108. The leads 104, 106, 108 are located at various locations of the heart 102, such as an atrium, a ventricle, or both, to measure cardiac signals of the heart 102. The leads 104, 106, 108 include the right ventricular (RV) lead 104, the right atrial (RA) lead 106, and the coronary sinus lead 108. Several electrodes 112, 114, 116, 118, 120, 122, 124, 126, 128 are coupled with the leads 104, 106, 108 for sensing cardiac signals and/or for delivering stimulus or stimulation pulses to the heart 102. The housing 110 may be one of the electrodes and is often referred to as the “can”, “case”, or “case electrode.”

The RV lead 104 is coupled with an RV tip electrode 122, an RV ring electrode 124, and an RV coil electrode 126. The RV lead 104 may include a superior vena cava (SVC) coil electrode 128. The right atrial lead 106 includes an atrial tip electrode 112 and an atrial ring electrode 114. The coronary sinus lead 108 includes a left ventricular (LV) tip electrode 116, a left atrial (LA) ring electrode 118 and an LA coil electrode 120. Alternatively, the coronary sinus lead 108 may be a quadropole lead that includes several electrodes disposed within the left ventricle. Leads and electrodes other than those shown in FIG. 1 may be included in the IMD 100 and positioned in or proximate to the heart 102.

The IMD 100 monitors cardiac signals of the heart 102 to determine if and when to deliver stimulus pulses to one or more chambers of the heart 102. The IMD 100 may deliver pacing stimulus pulses to pace the heart 102 and maintain a desired heart rate and/or shocking stimulus pulses to treat an abnormal heart rate such as tachycardia or bradycardia. As described above, the presence of an external magnetic field such as that of MR imaging system may cause magnetic forces to interfere with the operation of the IMD 100.

In order to avoid under- or over-sensing the cardiac signals when the IMD 100 is in the presence of a relatively large external magnetic field, the IMD 100 may switch modes of operation from a normal mode to a magnetic resonance (MR) safe mode when the IMD 100 enters the magnetic field. While in the MR safe mode, the IMD 100 may change the algorithms, software, or logical steps by which the cardiac signals are monitored to identify cardiac instability. For example, the IMD 100 may change which algorithms are used to identify an arrhythmia. Alternatively, the IMD 100 may cease measuring or sensing the cardiac signals. Once the IMD 100 leaves the magnetic field, the IMD 100 may switch back to the normal mode of operation. In the normal mode, the IMD 100 may resume monitoring the cardiac signals as the IMD 100 did before the IMD 100 entered the magnetic field.

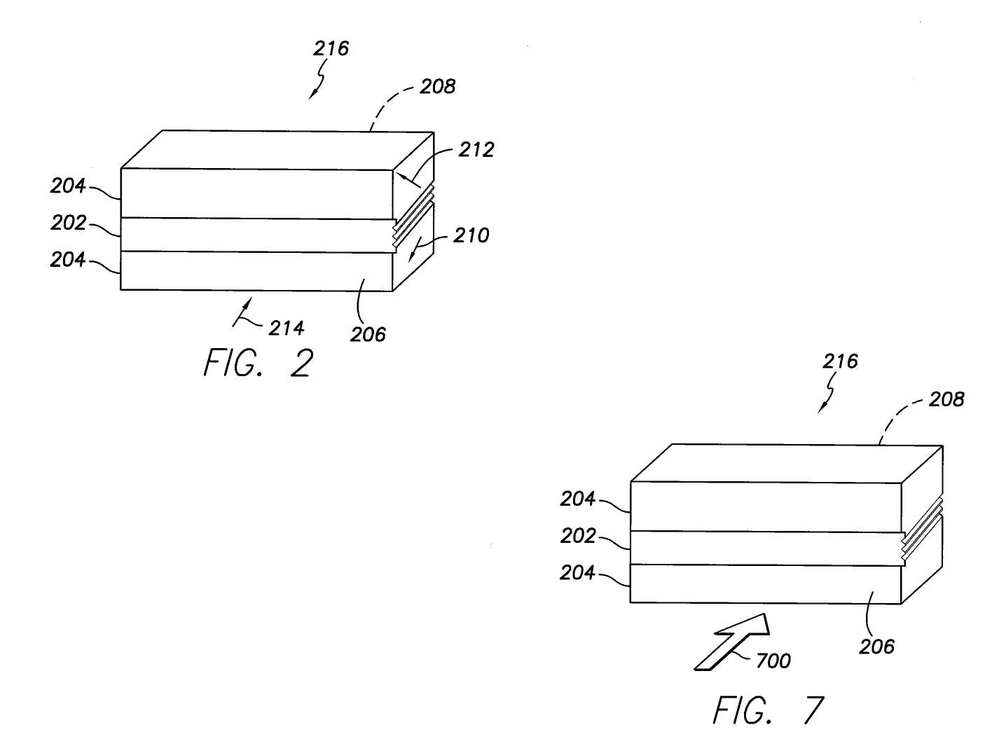

The IMD 100 includes a field measurement sensor 130 (shown schematically in FIG. 1) that is capable of measuring relatively large external magnetic fields to which the IMD 100 is exposed. The sensor 130 measures the magnetic fields to determine when to switch modes of operation. For example, the sensor 130 may measure an external magnetic field to determine if the IMD 100 should switch to an MR safe mode of operation. The sensor 130 also may measure the magnetic field to determine if the IMD 100 should switch back to the normal mode of operation. As described below, the sensor 130 includes a low permeability sensing resistive element 216 (shown in FIG. 2) that has an electrical resistance characteristic that changes when exposed to different external magnetic fields. The changing resistance of the resistive element 216 is used to sense, detect and/or differentiate between different external magnetic fields to which the sensor 130 is exposed in one embodiment.

FIG. 2 is a schematic illustration of a low permeability sensing resistive element 216 in accordance with one embodiment. The resistive element 216 is included in the sensor 130 (shown in FIG. 1). The resistive element 216 has an electrical resistance characteristic that changes based on the external magnetic fields to which the sensor 130 is exposed. Based on the changing resistance of the resistive element 216, the sensor 130 may be capable of measuring external magnetic fields of relatively large magnitudes, such as magnetic fields of greater than 0.2 Tesla. While the sensor 130 can be capable of measuring external magnetic fields of less than 0.2 Tesla, the sensor 130 may be able to measure external magnetic fields of greater than 0.2 Tesla with the resistive element 216 remaining unsaturated by the external magnetic fields. In various embodiments, the sensor 130 measures external magnetic fields of at least 0.5, 0.7, 1.0, 1.2, 1.5, 2.0, 2.5, or 3.0 Tesla without the resistive element 216 becoming saturated by the magnetic fields. For example, the sensor 130 may be sensitive to changes in a relatively large external magnetic field without the resistive element 216 being saturated by the magnetic field.

The resistive element 216 includes alternating layers of paramagnetic and conductive materials. In the illustrated embodiment, the resistive element 216 includes a conductive layer 202 that is sandwiched, or disposed between, paramagnetic layers 204. The conductive and paramagnetic layers 202, 204 extend from a front face 206 to a rear face 208. While the layers 202, 204 are shown as planar sheets, alternatively the layers 202, 204 may be disposed in a different arrangement. For example, the layers 202, 204 may be formed as concentric tubes or cylinders with the conductive layer 202 between the paramagnetic layers 204. Additionally, the number of layers 202, 204 in the illustrated embodiment is merely one example. The resistive element 216 may include a greater number of paramagnetic layers 204 and conductive layers 202.

The conductive layer 202 is formed from one or more conductive materials, such as metals or metal alloys. The conductive layer 302 may be a non-ferromagnetic or non-magnetic layer. For example, the conductive layer 202 may be formed from materials that do not independently generate a surrounding magnetic field and/or that do not independently generate a magnetic field when exposed to an external magnetic field. The conductive layer 202 is formed from copper (Cu) or copper alloys in one embodiment. The conductive layer 202 may be a relatively thin film. By way of example only, the conductive layer 202 may be one to three millimeters thick.

The paramagnetic layers 204 are formed from one or more paramagnetic materials. The paramagnetic materials forming the paramagnetic layers 204 generate magnetic fields when the materials are exposed to an external magnetic field but may not generate a magnetic field when the external magnetic field is removed. The magnetic fields generated by the paramagnetic materials may be relatively weak when compared to the external magnetic field to which the materials are exposed.

The paramagnetic layers 204 may be formed from low permeability paramagnetic materials that do not become saturated until exposed to very high external magnetic fields. In one embodiment, the paramagnetic layer 204 may only become saturated at very high external magnetic fields, such as 8.0 or 10.0 Tesla. The low permeability materials from which the paramagnetic layers 204 formed may be materials that are generally insensitive to low external magnetic fields but are sensitive to higher external magnetic fields. In one embodiment, the paramagnetic low permeability material may be a material having a relative magnetic permeability (μᵣ) of 1.0 to 1.3. The paramagnetic low permeability materials that may be included in the paramagnetic layers 204 may have a magnetic susceptibility characteristic (κ) that is related to the relative magnetic permeability (μᵣ) of the material as follows:

κ=μᵣ−1 (Eqn. 1)

In one embodiment, the magnetic susceptibility characteristic (K) of the materials that may be included in the paramagnetic layers 204 may be between −0.000121 to 0.3, inclusive. Another manner to describe the types of materials that may be included in the paramagnetic layers 204 is the magnetic mass susceptibility (ω). The magnetic mass susceptibility (χ) is related to the magnetic susceptibility characteristic (κ) and density of the materials and may be as follows:

χ=κ/δ (Eqn. 2)

where χ represents the magnetic mass susceptibility of the material, expressed in cubic centimeters per gram; κ is the magnetic susceptibility characteristic of the material; and δ represents the density of the material expressed in grams per cubic centimeter. In one embodiment, the materials that may be included in the paramagnetic layers 204 have a magnetic mass susceptibility (χ) of −0.000017 to 0.1, inclusive. The Table shown below includes a listing of example materials that may be used as the paramagnetic layers 204. The materials in the Table are examples and are not an exclusive list of all potential materials that may be included in the paramagnetic layers 204.

| Material | χ (cm³/gram) |
|---|---|
| Lanthanide, Neodymium | 0.000453 |
| Nd | |
| Lanthanide, | 0.000314 |
| Praseodymium Pr | |
| Antimony, Sb | −0.0000109 |
| Manganese Mn | 0.000148 |
| Beryllium Be | −0.0000126 |
| Titanium, Ti | 0.0000157 |
| Zinc, Zn | −0.00000197 |
| Rhenium, Re | 0.00000037 |
| Niobium, Nb | 0.0000189 |
| Vanadium V | 0.0000176 |
| Tantalum, Ta | 0.0000117 |
| Tungsten, W | 0.00000352 |

Other materials that may be used in the paramagnetic layers 204 include indium (In), lithium (Li), aluminum (Al), magnesium (Mg), sodium (Na), and the like. Other types of paramagnetic materials may be used.

The magnetic fields generated by the paramagnetic layers 204 are created by the alignment of magnetic dipole moments within the materials in the paramagnetic layers 204. A magnetic dipole moment is a magnetic field that is created by the spin of an electron. The magnetic dipole moment of an electron varies in direction due to the direction of spin of the electron. For example, an electron spinning about an axis in one direction may have a magnetic dipole moment that is oriented in a first direction while another electron spinning in an opposite direction may have a magnetic dipole moment that is oriented in an opposite second direction. The exposure of the paramagnetic layers 204 to an external magnetic field causes the magnetic dipole moments of the electrons in the paramagnetic layers 204 to be aligned with one another. As external magnetic field increases in strength, more magnetic dipole moments become aligned with each other. The alignment of the magnetic dipole moments of the electrons increases the strength of the magnetic field generated by the paramagnetic layer 204.

FIG. 13 is a schematic illustration of a low permeability sensing resistive element 1300 in accordance with another embodiment of the present disclosure. Similar to the resistive element 216 (shown in FIG. 2), the resistive element 1300 may be included in the sensor 130 (shown in FIG. 1). The resistive element 1300 has an electrical resistance characteristic that changes based on the external magnetic fields to which the sensor 130 is exposed. The resistive element 1300 includes alternating layers of paramagnetic and conductive materials. In the illustrated embodiment, the resistive element 1300 includes a conductive layer 1302 that is sandwiched, or disposed between, paramagnetic layers 1304, 1306.

In contrast to the resistive element 216, the paramagnetic and conductive layers 1302, 1304, 1306 form a pyramidal shape. For example, the paramagnetic layer 1306 forms the base of a three-dimensional pyramid and the paramagnetic layer 1304 forms the apex of the pyramid with the conductive layer 1302 forming the middle portion of the pyramid. The resistive element 1300 may include a greater number of paramagnetic layers 1304, 1306 and/or conductive layers 1302. The pyramidal shape shown in FIG. 13 is merely an example of the wide variety of shapes that the resistive element 1300 in the sensor 130 (shown in FIG. 1) may take. Other shapes may be used.

FIG. 14 is a schematic illustration of a low permeability sensing resistive element 1400 in accordance with another embodiment of the present disclosure. Similar to the resistive element 216 (shown in FIG. 2), the resistive element 1400 may be included in the sensor 130 (shown in FIG. 1). The resistive element 1400 has an electrical resistance characteristic that changes based on the external magnetic fields to which the sensor 130 is exposed. The resistive element 1400 includes alternating layers of paramagnetic and conductive materials. In the illustrated embodiment, the resistive element 1400 includes a conductive layer 1402 that is sandwiched, or disposed between, paramagnetic layers 1404. In contrast to the resistive element 216, the paramagnetic and conductive layers 1402, 1404 form a cylindrical shape. For example, the paramagnetic and conductive layers 1402, 1404 form a cylinder that is elongated from a front circular face 1406 to an opposite rear circular face 1408.

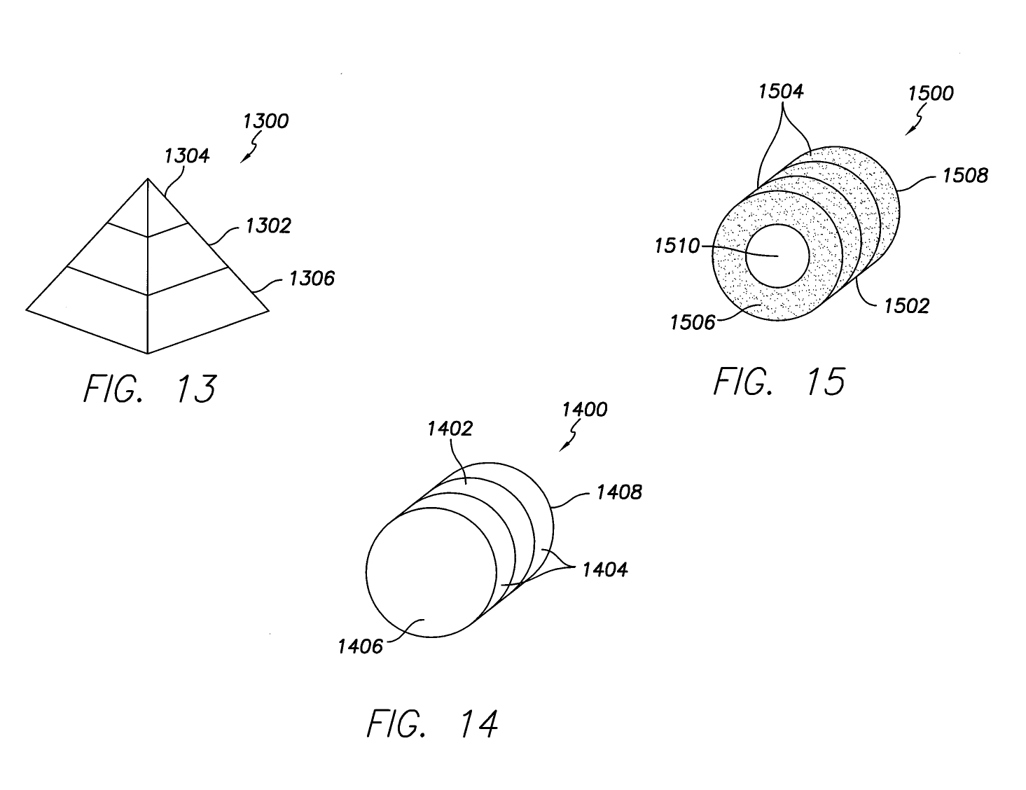

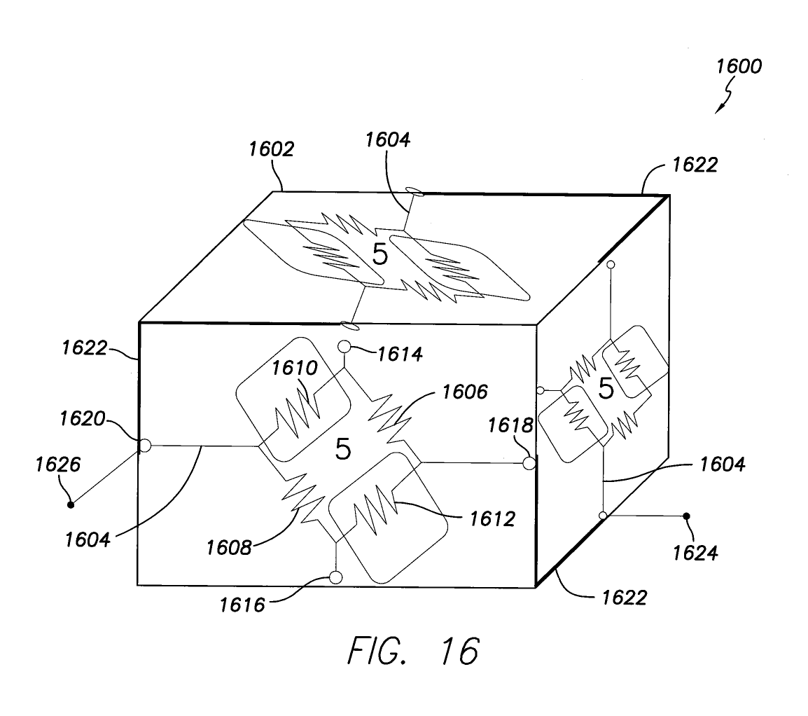

FIG. 15 is a schematic illustration of a low permeability sensing resistive element 1500 in accordance with another embodiment of the present disclosure. Similar to the resistive element 1400 (shown in FIG. 14), the resistive element 1500 may be included in the sensor 130 (shown in FIG. 1). The resistive element 1500 has an electrical resistance characteristic that changes based on the external magnetic fields to which the sensor 130 is exposed. The resistive element 1500 includes alternating layers of paramagnetic and conductive materials. In the illustrated embodiment, the resistive element 1500 includes a conductive layer 1502 that is sandwiched, or disposed between, paramagnetic layers 1504. In contrast to the resistive element 1400, the paramagnetic and conductive layers 1502, 1504 form a tubular shape. For example, the paramagnetic and conductive layers 1502, 1504 form a tube that is elongated from a front O-shaped face 1506 to an opposite rear O-shaped face 1508 with an opening or channel 1510 extending therethrough.

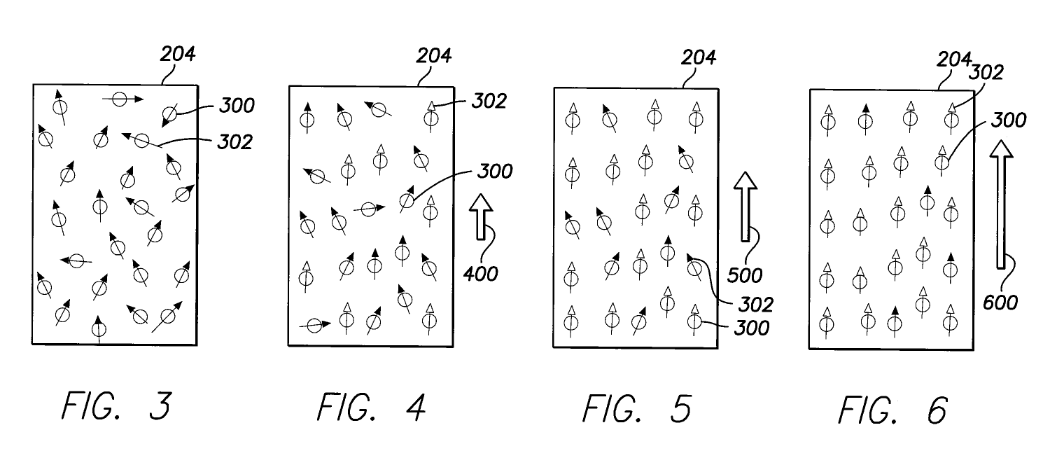

FIG. 3 illustrates a schematic diagram of magnetic dipole moments 302 of electrons 300 in one of the paramagnetic layers 204 in accordance with one embodiment. The magnetic dipole moments 302 are represented by arrows with the direction of the arrow indicative of the orientation of magnetic north of the magnetic dipole moment 302. The paramagnetic layer 204 shown in FIG. 3 is not exposed to an external magnetic field. The absence of an external magnetic field to align at least some of the magnetic dipole moments 302 results in the magnetic dipole moments 302 being approximately randomly oriented throughout the paramagnetic layer 204. Consequently, the paramagnetic layer 204 does not create a magnetic field in the absence of an external magnetic field, or creates a very weak magnetic field.

FIG. 4 illustrates a schematic diagram of the magnetic dipole moments 302 of the paramagnetic layer 204 shown in FIG. 3 when exposed to a first external magnetic field in accordance with one embodiment. In contrast to the paramagnetic layer 204 shown in FIG. 3, the paramagnetic layer 204 in FIG. 4 is exposed to an external magnetic field. By way of example only, the external magnetic field to which the paramagnetic layer 204 is exposed may have a strength of 0.7 Tesla. Alternatively, a different external magnetic field may be used. As shown in FIG. 4, a greater number of magnetic dipole moments 302 of the electrons 300 are aligned in a common direction when compared to the magnetic dipole moments 302 of FIG. 3. The magnetic dipole moments 302 may add together to create a net magnetization vector 400. The net magnetization vector 400 is shown as an arrow that represents the strength and direction of the magnetic field generated by the aligned magnetic dipole moments 302 in the paramagnetic layer 204. The direction of the net magnetization vector 400 indicates the direction in which the magnetic dipole moments 302 are aligned. The size or length of the net magnetization vector 400 relative to other net magnetization vectors 500, 600 (shown in FIGS. 5 and 6) represents the strength or magnitude of the magnetic field generated by the paramagnetic layer 204.

FIG. 5 illustrates a schematic diagram of the magnetic dipole moments 302 of the paramagnetic layer 204 shown in FIG. 4 when exposed to a second external magnetic field in accordance with one embodiment. The second external magnetic field is greater than the first external magnetic field described above. By way of example only, the external magnetic field to which the paramagnetic layer 204 is exposed in FIG. 5 may have a strength of 1.5 Tesla. As shown in FIG. 5, a greater number of magnetic dipole moments 302 are aligned with one another when compared to the magnetic dipole moments 302 of FIG. 4. As a result, the net magnetization vector 500 of the magnetic dipole moments 302 in FIG. 5 is greater than the net magnetization vector 400 (shown in FIG. 4). Consequently, the magnetic field generated by the paramagnetic layer 204 in the presence of the second external magnetic field is greater than the magnetic field generated by the paramagnetic layer 204.

FIG. 6 illustrates a schematic diagram of the magnetic dipole moments 302 of the paramagnetic layer 204 shown in FIG. 5 when exposed to a third external magnetic field in accordance with one embodiment. The third external magnetic field is greater than the second external magnetic field described above. By way of example only, the external magnetic field to which the paramagnetic layer 204 is exposed in FIG. 6 may have a strength of 3.0 Tesla. Similar to the comparison between FIGS. 4 and 5 above, a greater number of magnetic dipole moments 302 shown in FIG. 6 are aligned with one another when compared to the magnetic dipole moments 302 of FIG. 5. As a result, the net magnetization vector 600 of the magnetic dipole moments 302 in FIG. 6 is larger than the net magnetization vector 500 (shown in FIG. 5). The magnetic field generated by the paramagnetic layer 204 in the presence of the third external magnetic field is greater than the magnetic field generated by the paramagnetic layer 204 when exposed to the second external magnetic field of FIG. 5 or the first external magnetic field of FIG. 4.

As shown in FIGS. 3 through 6, the magnitude or strength of the magnetic fields created by the paramagnetic layers 204 increases with increasing external magnetic fields. The magnetic field generated by the paramagnetic layer 204 may continue to increase until the paramagnetic layer 204 becomes saturated. The paramagnetic layer 204 becomes saturated when a further increase in an external magnetic field does not result in an increase in the net magnetization vector 400, 500, 600 (shown in FIGS. 4 through 6). For example, the external magnetic field to which the paramagnetic layer 204 is exposed may be increased until all or substantially all of the magnetic dipole moments 302 of the paramagnetic layer 204 are aligned, or until a further increase in the external magnetic field does not result in more of the magnetic dipole moments 302 being aligned with one another.

Magnetic fields generated by the paramagnetic layers 204 in the resistive element 216 are used by the sensor 130 (shown in FIG. 1) to measure an external magnetic field to which the sensor 130 is exposed. Changes in the magnetic fields created by the paramagnetic layers 204 vary an electrical resistance characteristic of the conductive layer 202 in the resistive element 216. For example, an increase in the magnetic fields generated by the paramagnetic layers 204 may reduce the electrical resistance of the conductive layer 202 between the front and rear face 206, 208. Conversely, a decrease in the magnetic fields created by the paramagnetic layers 204 may increase the electrical resistance of the conductive layer 202.

The electrical resistance of the conductive layer 202 changes due to variances in the net magnetization vectors 400, 500, 600 (shown in FIGS. 4 through 6) of the paramagnetic layers 204. When exposed to a relatively low external magnetic field 214 or in the absence of an external magnetic field 214, each of the paramagnetic layers 204 may have a relatively small or nonexistent net magnetization vector 210, 212. The net magnetization vectors 210, 212 may be very small or nonexistent due to the approximately random orientation of the magnetic dipole moments 302 (shown in FIG. 3) in the paramagnetic layers 204 when the external magnetic field 214 is very small or nonexistent.

As described above, the conductive layer 202 may be relatively thin. A thin layer of conductive material between the paramagnetic layers 204 may have a relatively large electrical resistance due to the scattering of electrons in the conductive material that is caused by the random orientations of the magnetic dipole moments 302 (shown in FIG. 3) in the paramagnetic layers 204. For example, the electrons in the relatively thin conductive layer 202 may be more accessible or susceptible to the randomly oriented magnetic dipole moments 302 in the paramagnetic layers 204. The volume of space encompassed by the magnetic field of each magnetic dipole moment 302 is relatively small. But, if the thickness of the conductive layer 202 is sufficiently thin, then the magnetic fields of the magnetic dipole moments 302 may encompass at least some of the electrons in the conductive layer 202. The electrons in the conductive layer 202 that are exposed to the magnetic dipole moments 302 in the paramagnetic layers 204 may be scattered or moved by the magnetic dipole moments 302.

If the magnetic dipole moments 302 (shown in FIG. 3) are randomly oriented in the paramagnetic layers 204, then the electrons in the conductive layer 202 are scattered in random directions. As a result, the electrons in the conductive layer 202 are scattered in different, non-parallel directions due to the different orientations of the net magnetization vectors 210, 212. The electrical resistance of the conductive layer 202 is related to or based on the mean free path of electrons in the conductive layer 202 that are used to conduct the current through the conductive layer 202. The scattering of the electrons in random or non-parallel directions in the conductive layer 202 decreases the mean free path of these electrons. As the mean free path of the electrons decreases, there is less distance or room for electrical conduction of available electrons in the conductive layer 202. As the mean free path for electrical conduction decreases, the electrical resistance of the conductive layer 202 increases.

FIG. 7 is a schematic illustration of the resistive element 216 when exposed to a relatively large external magnetic field 700 in accordance with one embodiment. The external magnetic field 700 is oriented in a direction toward the back face 208 of the resistive element 216. For example, the external magnetic field 700 may be oriented with magnetic north of the external magnetic field 700 toward the back face 208 and magnetic south of the external magnetic field 700 disposed toward the front face 206.

As described above, the external magnetic field 700 causes more of the magnetic dipole moments 302 (shown in FIG. 3) in the paramagnetic layers 204 to be aligned with one another. The alignment of the magnetic dipole moments 302 generates net magnetization vectors 702, 704 that are representative of the magnetic fields generated by the paramagnetic layers 204. Alternatively, the external magnetic field 700 may be angled or parallel to the front and back faces 206, 208. The illustrated net magnetization vectors 702, 704 are shown as parallel vectors oriented in opposite directions. For example, the net magnetization vectors 702, 704 are approximately parallel to one another or are not oriented in transverse or angled directions with respect to one another. Alternatively, the net magnetization vectors 702, 704 may be parallel or non-transverse and oriented in a common direction. In another example, the net magnetization vectors 702, 704 may be oriented toward the front and/or back faces 206, 208 of the resistive element 216.

The parallel orientation of the net magnetization vectors 702, 704 increases the mean free path of the electrons that conduct the electric current through the conductive layer 202 from the front face 206 to the back face 208. The net magnetization vectors 702, 704 are larger in magnitude than the net magnetization vectors 210, 212. Therefore, the net magnetization vectors 210, 212 exert larger magnetic forces on the electrons in the conductive layer 202. In contrast to the random scattering of the electrons caused by the net magnetization vectors 210, 212 (shown in FIG. 2), the parallel or non-transverse net magnetization vectors 702, 704 do not cause random scattering of the electrons in the conductive layer 202. As a result, the electrons that conduct the electric current in the conductive layer are not randomly scattered by the net magnetization vectors 702, 704. By reducing the scattering of electrons in the conductive layer 202, the mean free path of the electrons in the conductive layer 202 is increased and the electrical resistance of the conductor layer decreases.

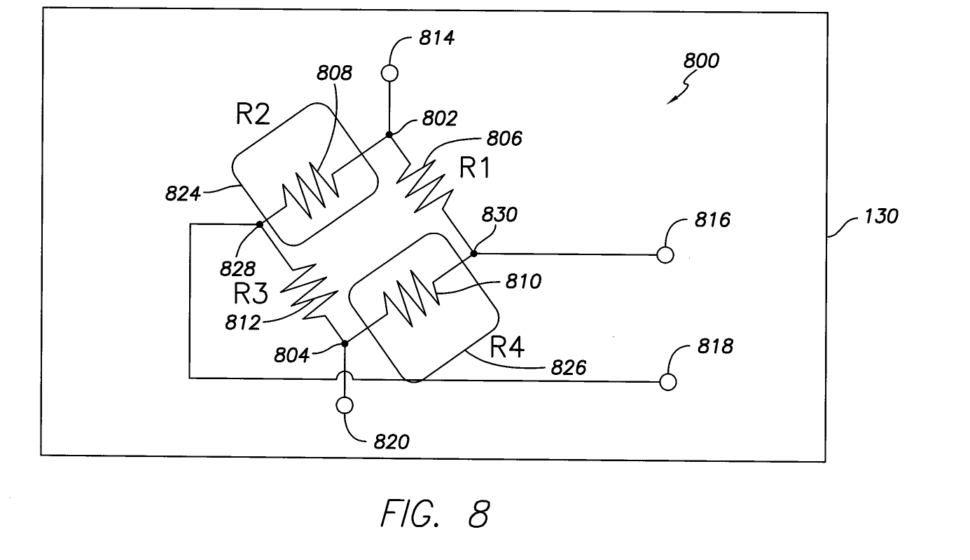

FIG. 8 is a circuit diagram of the sensor 130 in accordance with one embodiment. The sensor 130 includes a field measurement circuit 800 that is used to measure the external magnetic field 700 (shown in FIG. 7). The circuit 800 is used to measure a differential voltage across two nodes 816, 818 of the circuit 800. This differential voltage is proportional to the strength of the external magnetic field 700. By way of example only, the differential voltage may be measured by a galvanometer or other device. The differential voltage may be referred to as a field measurement of the external magnetic field 700. Alternatively, the field measurement of the external magnetic field 700 may be another measurement based on the changing electrical resistance of the conductive layers 202 (shown in FIG. 2) of low permeability sensing resistive elements 808, 810.

A power source, such as a battery 1154 (shown in FIG. 11), supplies power or voltage to the circuit 800 across two nodes 814, 820. In the illustrated embodiment, the circuit 800 includes a Wheatstone Bridge circuit between the nodes 814, 820. The Wheatstone Bridge circuit includes four resistive elements 806, 808, 810, 812. In the illustrated embodiment, the resistive elements 808, 810 are low permeability sensing resistive elements 808, 810. Each of the resistive elements 808, 810 may be similar to the resistive element 200 (shown in FIG. 2). For example, each resistive element 808, 810 may include one or more conductive layers 202 (shown in FIG. 2) with an electrical resistance characteristic that varies based on the external magnetic field 700. In one embodiment, each of the resistive elements 808, 810 is enclosed in an electromagnetic shield 824, 826.

The resistive elements 806, 812 may be reference resistive elements 806, 812 that have a predetermined or known electrical resistance characteristic. The electrical resistance of the resistive elements 806, 812 may not change based on the exposure of the sensor 130 to an external magnetic field.

The Wheatstone Bridge in the circuit 800 extends between nodes 802, 804 and between nodes 828, 830. The node 814 is joined with the node 802 and the node 820 is joined with the node 804 such that power is supplied to the circuit 800 across the nodes 814, 820 and to the Wheatstone Bridge across the nodes 802, 804. The node 818 is joined to the node 828 and the node 816 is joined to the node 830 such that the differential voltage measured across the nodes 816, 818 of the circuit 800 is approximately the same as the differential voltage across the nodes 828, 830. The resistive element 806 is located between the nodes 802 and 830 and the resistive element 812 is located between the nodes 828 and 804. The resistive element 808 is located between the nodes 802 and 828 and the resistive element 810 is located between the nodes 804 and 830. The node 828 is located between the resistive element 808 and the resistive element 812. The node 830 is located between the resistive element 806 and the resistive element 810. The Wheatstone Bridge includes two parallel pathways, or “legs.” One of the legs includes the resistive elements 808, 812 in series with one another. The other of the legs includes the resistive elements 806, 810 in series with one another.

In operation, the power source provides current to the circuit 800 across the nodes 814, 820. The current is distributed between the two legs of the Wheatstone Bridge. Based on the electrical resistances of the resistive elements 806, 810 in one leg and the resistive elements 808, 812 in the other leg, a voltage differential between the legs may exist across the nodes 816, 818.

In the Wheatstone Bridge, if no voltage differential exists across the nodes 816, 818, then the ratio of electrical resistances in each leg of the Wheatstone Bridge may be equal. For example, if the electrical resistance of the resistive element 806 is represented as R₁, the electrical resistance of the resistive element 810 is represented as R₄, the electrical resistance of the resistive element 808 is represented as R₂, and the electrical resistance of the resistive element 812 is represented as R₃, then the following relationship may apply when no voltage difference or drop exists across the nodes 816, 818:

$$\frac{{R}_{1}}{{R}_{4}} = \frac{{R}_{2}}{{R}_{3}} \qquad ( Eqn . 3 )$$

But, the electrical resistances of the resistive elements 808, 810 may change based on the external magnetic field 700 (shown in FIG. 7). In one embodiment, the electrical resistance of the resistive elements 806, 812 are approximately the same and the electrical resistances of the resistive elements 808, 810 are approximately the same when exposed to an external magnetic field 700. As the strength of the external magnetic field 700 changes, the ratio of electrical resistances in each leg of the Wheatstone Bridge may no longer be equal. For example, as the electrical resistances R₂ and R₄ of the resistive elements 808, 810 increase, the ratio of the electrical resistance R₁ of the resistive element 806 to the electrical resistance R₄ of the resistive element 810 may not be equal to the ratio of the electrical resistance R₂ of the resistive element 808 to the electrical resistance R₃ of the resistive element 812. As a result, a voltage differential may exist across the nodes 816, 818. This voltage differential may represent the strength of the external magnetic field 700. The voltage differential may increase as the strength of the external magnetic field 700 increases and decrease as the external magnetic field 700 decreases.

FIG. 16 is a schematic illustration of a field measurement sensor 1600 in accordance with another embodiment of the present disclosure. The sensor 1600 is shown as having a cuboid body 1602. Alternatively, the body 1602 may have a different three-dimensional shape. The sensor 1600 includes a field measurement circuit 1604 disposed on several of the faces of the body 1602. For example, the sensor 1600 may include a field measurement circuit 1604 on each of three orthogonal faces that intersect one another on the body 1602. Alternatively, the sensor 1600 may include a different number of circuits 1604. By way of example only, the sensor 1600 may have a circuit 1604 on each of the six faces of the body 1602.

Each of the circuits 1604 may be similar to the field measurement circuit 800 (shown in FIG. 8). For example, the circuits 1604 may include Wheatstone bridges that have two reference resistive elements 1606, 1608 and two sensing resistive elements 1610, 1612 with electrical resistance characteristics that change based on an external magnetic field. Power may be supplied to each of the circuits 1604 by a power source such as the battery 1154 (shown in FIG. 11). The power is supplied across nodes 1614, 1616 of each circuit 1604. The nodes 1614, 1616 may be similar to the nodes 814, 820 (shown in FIG. 8) of the circuit 800. A differential voltage may be obtained across nodes 1618, 1620 of each circuit 1604 when power is supplied to the circuit 1604. The nodes 1618, 1620 may be similar to the nodes 816, 818 (shown in FIG. 8) of the circuit 800 such that the differential voltage across the nodes 1618, 1620 may be used as a field measurement.

The nodes 1618, 1620 of different circuits 1604 may be electrically coupled to provide an additive field measurement across the circuits 1604. For example, the node 1618 of one circuit 1604 may be joined with the node 1620 of another circuit 1604 by a conductive pathway 1622. The circuits 1604 may be joined in series in this way. The node 1618 of the circuit 1604 on one end of the series of circuits 1604 may be coupled with a measurement node 1624 and the node 1620 of the circuit 1604 on the opposite end of the series of circuits 1604 may be coupled with a measurement node 1626. The connection of the circuits 1604 in series between the measurement nodes 1622, 1624 may cause the differential voltage of each circuit 1604 across the nodes 1618, 1620 of each circuit 1604 to be combined. The combined differential voltages may be measured across the measurement nodes 1622, 1624 to provide a summed differential voltage. The summed differential voltage may be used as the field measurement of an external magnetic field to which the sensor 1600 is exposed. The location of the circuits 1604 on orthogonal faces of the body 1602 may result in a sensor 1600 that is able to more reliably measure the strengths of external magnetic fields regardless of the orientation of the sensor 1600 with respect to the external magnetic field. For example, each of the circuits 1604 may be orthogonal to an x, y, or z direction of an external magnetic field such that at least one of the circuits 1604 is able to measure the strength of the external magnetic field irrespective of the orientation of the external magnetic field with respect to the faces of the body 1602.

In one embodiment, the sensor 130 (shown in FIG. 1) may include several sensors 1600 joined in series with one another. For example, the measurement nodes 1622, 1624 of two or more sensors 1600 may be joined in series such that the differential voltage measured across the measurement nodes 1622, 1624 of each circuit 1604 is added together and provided as a field measurement of the external magnetic field.

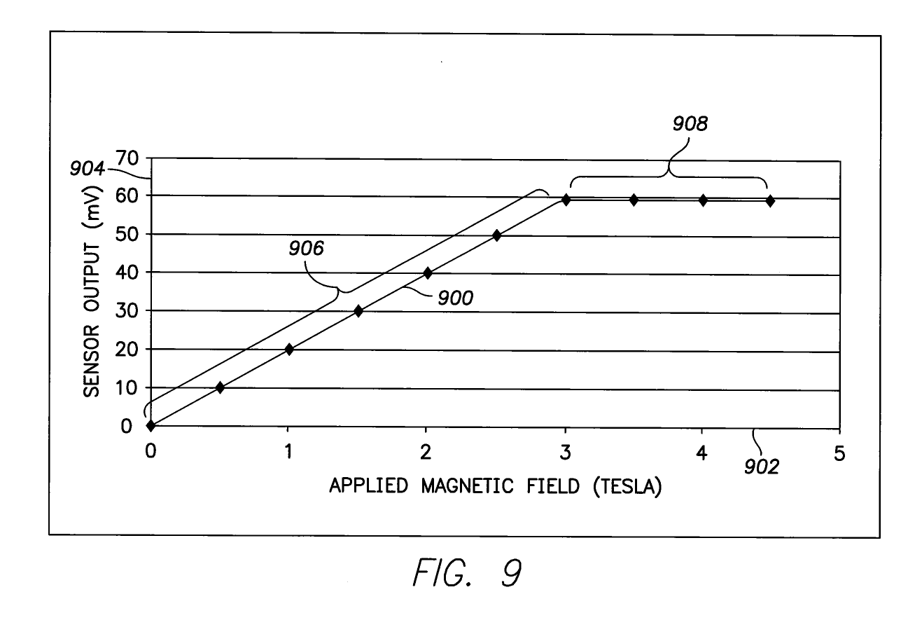

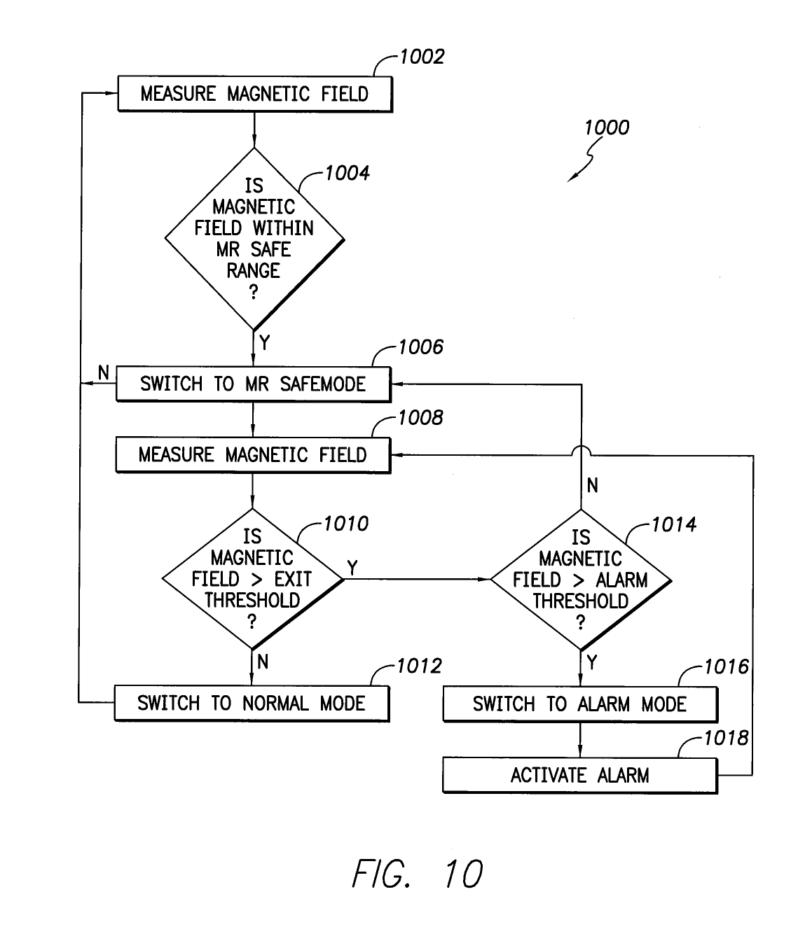

FIG. 9 illustrates an example of a relationship 900 between a voltage differential measured by the galvanometer 822 (shown in FIG. 8) and the strength of an external magnetic field 700 (shown in FIG. 7) in accordance with one embodiment. The relationship 900 is shown alongside a horizontal axis 902 representative of the external magnetic field 700 and a vertical axis 904 representative of the voltage differential measured between the nodes 816, 818 (shown in FIG. 8) of the sensing circuit 804 (shown in FIG. 8).

The relationship 900 includes an unsaturated section 906 and a saturated section 908. The unsaturated section 806 represents a dynamic relationship between the external magnetic field 700 (shown in FIG. 7) and the differential voltage, measured by the galvanometer 822 (shown in FIG. 8). In the illustrated embodiment, the unsaturated section 806 represents a linear relationship between the external magnetic field 700 and the differential voltage. Alternatively, the unsaturated section 806 may represent a different relationship between the external magnetic field 700 and the field measurement. For example, the relationship may be an exponential or other non-linear relationship. The differential voltage increases when the electrical resistances of the resistive elements 808, 810 (shown in FIG. 8) in the sensing circuit 804 (shown in FIG. 8) decrease. Therefore, as the external magnetic field 700 increases in strength, the electrical resistances of the resistive elements 808, 810 decrease.

The unsaturated section 906 extends along the horizontal axis 902 from zero Tesla to approximately 3.0 Tesla. The linear relationship between the field measurement and the external magnetic field 700 (shown in FIG. 7) indicates that the resistive elements 808, 810 (shown in FIG. 8) are sensitive to changes in the external magnetic field 700 from zero Tesla to 3.0 Tesla. A dynamic sensing range of each of the resistive elements 808, 810 represents the scope of external magnetic fields 700 over which the resistive elements 808, 810 remain in an unsaturated state. For example, the dynamic sensing range may be the range of external magnetic fields 700 over which the paramagnetic layers 204 (shown in FIG. 2) of the resistive elements 808, 810 are in an unsaturated state.

The saturated section 908 represents a saturation range of the resistive elements 808, 810 (shown in FIG. 8). For example, the saturated section 908 represents the range of external magnetic fields 700 (shown in FIG. 7) over which the resistive elements 808, 810 no longer have electrical resistance characteristics that increase in response to an increase in an external magnetic field. The resistive elements 808, 810 may become saturated when further increases in the external magnetic field 700 no longer result in corresponding increases in the net magnetization vectors 400, 500, 600 (shown in FIGS. 4 through 6) of the paramagnetic layers 204 (shown in FIG. 2). In contrast with the dynamic sensing range of the resistive elements 808, 810, the sensor 130 may be unable to measure changes in the external magnetic field 700 in the saturation range.

In the illustrated embodiment, the dynamic sensing range of the sensor 130 that includes the resistive elements 808, 810 extends up to 3.0 Tesla, at which point the resistive elements 808, 810 become saturated. Alternatively, the dynamic sensing range may increase to larger magnitudes of external magnetic fields 700 (shown in FIG. 7), with the resistive elements 808, 810 not being saturated until exposed to larger external magnetic fields 700. Conversely, in another embodiment the resistive elements 808, 810 may become saturated when exposed to lesser external magnetic fields 700. By way of example only, the sensors 808, 810 may not become saturated when exposed to external magnetic fields of up to 0.5, 0.7, 1.5, 2.0, or 3.0 Tesla.

The dynamic sensing range of the sensor 130 (shown in FIG. 1) may be controlled or adjusted by varying the paramagnetic materials used in the paramagnetic layers 204 (shown in FIG. 2) of the resistive elements 808, 810. Using materials with lower magnetic permeabilities may avoid saturating the resistive elements 808, 810 when the sensor 130 is exposed to larger external magnetic fields 700 (shown in FIG. 7). As the magnetic permeability of the material(s) used in the paramagnetic layers 204 decreases, the resistive elements 808, 810 are able to have electrical resistance characteristics that change in response to increases in larger external magnetic fields 700. Conversely, as the magnetic permeability of the paramagnetic layers 204 increases, the resistive elements 808, 810 may saturate at lower external magnetic fields 700.

The dynamic sensing range of the sensor 130 (shown in FIG. 1) may be controlled or adjusted by varying the thickness of the conductive layer 202 (shown in FIG. 2) of the resistive elements 808, 810. Increasing the thickness of the conductive layer 202 may permit the resistive elements 808, 810 to be exposed to larger external magnetic fields without being saturated, thereby causing the sensor 130 to be sensitive to increases in larger external magnetic fields 700 (shown in FIG. 7). For example, increasing the thickness of the conductive layer 202 may result in fewer electrons in the conductive layer 202 being affected or scattered by the magnetic dipole moments 302 (shown in FIG. 3) or net magnetization vectors 400, 500, 600 (shown in FIGS. 4 through 6). The electrons in the conductive layer 202 that are located at or near the interfaces between the conductive layer 202 and each paramagnetic layer 204 may be scattered by the magnetic dipole moments 302 while the electrons located away from the conductive layer 202 and toward the center or middle of the thickness of the conductive layer 202 may be unaffected by the magnetic dipole moments 302 or net magnetization vectors 400, 500, 600. As a result, the external magnetic field 700 may have a lesser effect on the mean free path of the electrons that conduct current through the conductive layer 202 relative to thinner conductive layers 202. The sensor 130 may be able to measure larger external magnetic fields 700 before the resistive elements 808, 810 become saturated.

Reducing the thickness of the conductive layer 202 (shown in FIG. 2) may cause the resistive elements 808, 810 (shown in FIG. 8) to be saturated when exposed to lesser external magnetic fields 700 (shown in FIG. 7). For example, a thinner conductive layer 202 permits more of the electrons in the conductive layer 202 to be scattered by the magnetic dipole moments 302 (shown in FIG. 3) and/or net magnetization vectors 400, 500, 600 (shown in FIGS. 4 through 6) when exposed to smaller external magnetic fields 700. As a result, a smaller external magnetic field 700 may saturate the resistive elements 808, 810 when the thickness of the conductive layer 202 is decreased.

The dynamic sensing range of the sensor 130 (shown in FIG. 1) may be controlled or adjusted by varying the thickness of the paramagnetic layers 204 (shown in FIG. 2). Increasing the thicknesses of the paramagnetic layers 204 may permit the sensors 808, 810 to have electrical resistance characteristics that change in response to changes in larger external magnetic fields 700 (shown in FIG. 7). For example, increasing the thickness of the paramagnetic layers 204 may result in a greater number of electrons 300 (shown in FIG. 3) with corresponding magnetic dipole moments 302 (shown in FIG. 3) being provided. As the number of electrons 300 and magnetic dipole moments 302 in the paramagnetic layers 204 increases, the paramagnetic layers 204 are able to be exposed to larger external magnetic fields 700. As described above, the paramagnetic layers 204 may become saturated when all or substantially all of the magnetic dipole moments 302 are aligned. Increasing the number of magnetic dipole moments 302 increases the magnitude of the external magnetic field 700 at which the paramagnetic layers 204 are saturated. Conversely, decreasing the thicknesses of the paramagnetic layers 204 decreases the number of magnetic dipole moments 302 in the layers 204. As a result, the paramagnetic layers 204 may be saturated at lower external magnetic fields 700.

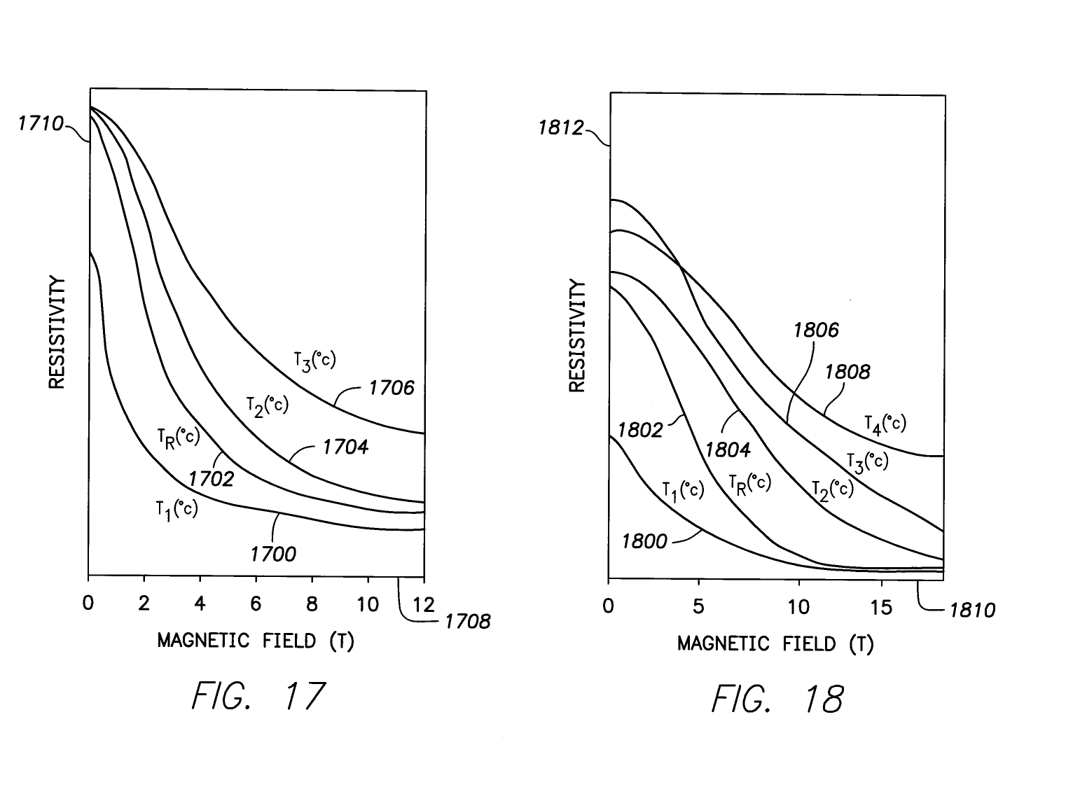

FIGS. 17 and 18 illustrate several relationships 1700, 1702, 1704, 1706, 1800, 1802, 1804, 1806, 1808 between electrical resistivity of the resistive elements 808, 810 (shown in FIG. 8) and external magnetic fields in accordance with one embodiment. The relationships 1700, 1702, 1704, 1706, 1800, 1802, 1804, 1806, 1808 are shown alongside horizontal axes 1708, 1810 that represent external magnetic fields expressed in Teslas and vertical axes 1710, 1812 that represent the volume resistivities of the resistive elements 808, 810 expressed in ohms*meters.

The different relationships 1700, 1702, 1704, 1706, 1800, 1802, 1804, 1806, 1808 represent the electrical resistivities of the resistive elements 808, 810 (shown in FIG. 8) at different temperatures. For example, the relationships 1702 and 1802 represent the electrical resistivities of the resistive elements 808, 810 at room temperature, or at approximately 20 degrees Celsius. The relationships 1700 and 1800 represent the electrical resistivities of the resistive elements 808, 810 at a temperature below room temperature. The relationships 1704 and 1804 represent the electrical resistivities of the resistive elements 808, 810 at a temperature above room temperature. The relationships 1706 and 1806 represent the electrical resistivities of the resistive elements 808, 810 at a temperature above room temperature and above the temperature represented by the relationships 1704, 1804. The relationship 1808 represents the electrical resistivities of the resistive elements 808, 810 at a temperature above room temperature and above the temperature represented by the relationship 1806.

As shown in the relationships 1700, 1702, 1704, 1706, 1800, 1802, 1804, 1806, 1808, the electrical resistivity of the resistive elements 808, 810 decreases with increasing external magnetic field strengths. As described above, the changing relationship between resistivity and external magnetic field strength may be used by the IMD 100 (shown in FIG. 1) to identify and distinguish between different external magnetic fields to which the IMD 100 may be exposed. In one embodiment, the dependence of the resistivity of the resistive elements 808, 810 (shown in FIG. 8) may be used to distinguish between different external magnetic fields. For example, the IMD 100 may use one or more of the relationships 1700, 1702, 1704, 1706, 1800, 1802, 1804, 1806, 1808 to identify the strength of an external magnetic field based on a temperature of the resistive elements 808, 810. The temperature of the resistive elements 808, 810 may be obtained using a sensor or other device capable of determining the temperature of the resistive elements 808, 810 and/or of the environment surrounding the resistive elements 808, 810.

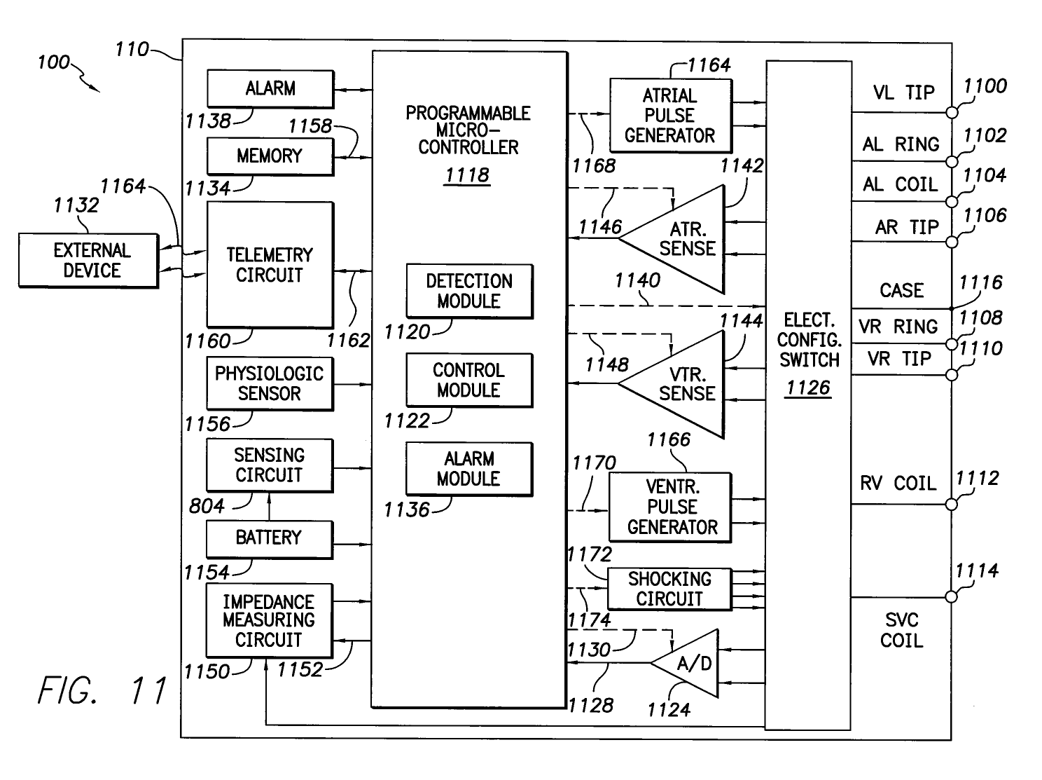

FIG. 10 is a flowchart of a method 1000 for switching modes of an IMD 100 (shown in FIG. 1) based on a magnetic field in accordance with one embodiment. The method 1000 may be performed while the IMD 100 is sensing and/or analyzing cardiac signals. For example, the method 1000 may occur in parallel with the obtaining and monitoring of a patient's cardiac signals.

At 1002, an external magnetic field is measured. The external magnetic field is the magnetic field to which the IMD 100 (shown in FIG. 1) is exposed. As described above, a field measurement may be obtained from the sensor 130 (shown in FIG. 1) of the IMD 100. The field measurement may be a voltage differential measured using the circuit 800 (shown in FIG. 8) of the sensor 130 and that is based on the electrical resistances of one or more low permeability sensing resistive elements 808, 810 (shown in FIG. 8). The field measurement may be obtained at a predetermined sampling rate. By way of example only, the field measurement may be obtained at a rate of 50 KHz or higher. Alternatively, a different sampling rate may be used.

At 1004, the field measurement of the external magnetic field is compared to an MR safe range. The MR safe range is a predetermined range of field measurements that extend from a lower threshold to an upper threshold of magnetic field strengths. The MR safe range may correspond to a range of external magnetic fields that the IMD 100 (shown in FIG. 1) may continue to safely operate within after switching to an MR safe mode of operation. For example, the strengths of the external magnetic fields in the MR safe range may be sufficiently low that the IMD 100 may continue safely operating while exposed to the external magnetic fields, but sufficiently high that the mode of operation of the IMD 100 must be adjusted or changed to prevent over-sensing or under-sensing cardiac events. In one embodiment, the MR safe range extends between 1.3 Tesla and 1.6 Tesla. However, the MR safe range may have a different upper and/or lower threshold. For example, the MR safe range may have a lower threshold of 0.5 Tesla, 0.7 Tesla, 1.0 Tesla, 1.5 Tesla, 2.0 Tesla, 2.5 Tesla, or 3.0 Tesla. The MR safe range may have an upper threshold of 3.0 Tesla, 3.5 Tesla, 4.0 Tesla, 6.0 Tesla, 8.0 Tesla, or 10.0 Tesla.

In one embodiment, the IMD 100 (shown in FIG. 1) samples the field measurements obtained by the field measurement sensor 130 (shown in FIG. 1). As described above, the field measurements may be a differential voltage output from one or more sensors 130 that corresponds to the strength of the external magnetic field. The IMD 100 may calculate a moving average of the field measurements and compare the moving average to the MR safe range. In one embodiment, the IMD 100 calculates a moving average of the most recent five samples of the field measurements obtained by the sensor 130. Alternatively, a different number of field measurements may be included in the average. In another embodiment, the IMD 100 calculates another characteristic of the field measurements. For example, the IMD 100 may calculate a mean, deviation, maximum, minimum, and the like, of the field measurements to compare to the MR safe range.

If the sampled field measurements fall within the MR safe range, then the field measurements may indicate that the IMD 100 (shown in FIG. 1) is exposed to a relatively large external magnetic field that is generated by an MR imaging system. As a result, at 1006, the IMD 100 switches modes of operation from a normal operating mode to an MR safe mode. Alternatively, if the sampled field measurements do not fall within the MR safe range, then the field measurements may indicate that the IMD 100 is not within the proximity of an active MR imaging system or an external magnetic field generated by the MR imaging system. For example, the field measurements may be below the lower threshold of the MR safe range. Consequently, flow of the method 1000 returns to 1002 where the IMD 100 continues to obtain measurements of external magnetic fields to which the IMD 100 may be exposed. The IMD 100 may continue to sense and monitor cardiac signals while sampling the external magnetic field strengths in order to provide approximately continuous monitoring for relatively large external magnetic fields.

In another embodiment, the field measurement may be compared to several thresholds to distinguish between multiple MR magnetic field strengths. For example, several different thresholds may be used to define different MR magnetic field strength ranges. The field measurement of an external magnetic field is compared to the different thresholds to determine which range of magnetic field strengths encompasses the field measurement.

At 1006, the IMD 100 (shown in FIG. 1) switches the mode of operation of the IMD 100 from the normal mode to the MR safe mode. In the MR safe mode, the IMD 100 may change one or more of the algorithms, processes, methods, analyses, and the like, that are used to sense and monitor the cardiac signals of the heart 102 (shown in FIG. 1). For example, the IMD 100 may switch to a VOO mode when entering the MR safe mode. In the VOO mode, the IMD 100 stops sensing the cardiac signals of the heart 102 and paces the heart 102 at a predetermined rate. The IMD 100 may pace a ventricle at a fixed, predetermined lower rate interval without regard to the cardiac signals. The IMD 100 does not monitor or respond to any cardiac events that otherwise would be identified based on the cardiac signals. For example, the IMD 100 may ignore any cardiac signals that are indicative of a cardiac event when in the VOO mode and continue pacing the ventricle at the rate interval.

In another example, the IMD 100 (shown in FIG. 1) may switch to an AOO mode. In the AOO mode, the IMD 100 stops sensing the cardiac signals of the heart 102 (shown in FIG. 1) and paces one or both of the atria the heart 102 at a predetermined rate without regard to the cardiac signals. The IMD 100 does not monitor or respond to any cardiac events that otherwise would be identified based on the cardiac signals. Alternatively, the IMD 100 may switch to a DOO mode. In the DOO mode, the IMD 100 stops sensing and monitoring the cardiac signals. The IMD 100 paces both an atrium and a ventricle of the heart 102 at a fixed, predetermined rate. For example, the IMD 100 may apply stimulus pulses to an atrium at a first rate and, after a predetermined delay following each stimulus pulse to the atrium, apply a stimulus pulse to a ventricle. In another embodiment, the IMD 100 may continue to sense and monitor cardiac signals in the MR safe mode using one or more algorithms that differ from the algorithms used in the normal mode.

In another embodiment, the IMD 100 may switch modes from the current mode of operation of the IMD 100 to a different mode based on which of several MR magnetic field strength ranges includes the field measurement. As described above, the field measurement may be compared to several ranges of external magnetic fields to determine which of the ranges includes the field measurement. The different ranges may be associated with different operating modes of the IMD 100. The different operating modes may, in turn, be associated with different algorithms, processes, methods, analyses, and the like, that are used to sense and monitor the cardiac signals of the heart 102 (shown in FIG. 1). Based on the range that encompasses the field measurement, the IMD 100 may switch to a corresponding mode of operation and switch the methods used to sense and monitor signals of the heart 102.

At 1008, the IMD 100 (shown in FIG. 1) measures the external magnetic field to which the IMD 100 may continue to be exposed. For example, after entering an external magnetic field of sufficient strength to cause the IMD 100 to switch to an MR safe mode, the IMD 100 may continue measuring the strength of the external magnetic field in order to determine if the strength of the external magnetic field increases (such as by the IMD 100 entering a stronger external magnetic field or moving closer to the source of the external magnetic field) or if the strength of the external magnetic field decreases (such as by the patient leaving the proximity of the source of the external magnetic field).

At 1010, the IMD 100 (shown in FIG. 1) determines if the strength of the external magnetic field exceeds an exit threshold. The exit threshold is a predetermined lower limit of an external magnetic field. The exit threshold may be sufficiently low to represent the outer geographic boundary or edge of an external magnetic field generated by an MR imaging system. For example, the exit threshold may be 0.05 Tesla. Alternatively, the exit threshold may be a different limit, such as 0.02 Tesla, 0.5 Tesla, and the like. If the IMD 100 determines that the strength of the external magnetic field generated by the MR imaging system has decreased below the exit threshold, then the decrease in the external magnetic field may indicate that the IMD 100 is no longer in the vicinity of the MR imaging system or that the MR imaging system is no longer generating the external magnetic field. As a result, at 1012, the IMD 100 may switch back to the normal mode of operation. For example, the IMD 100 may resume sensing and monitoring the cardiac signals of the heart 102 (shown in FIG. 1) using the same algorithms, processes, methods, analyses, and the like that were used by the IMD 100 prior to entering the MR safe mode. Flow of the method 1000 may proceed back to 1002, where the IMD 100 measures the strength of any external magnetic field to which the IMD 100 may be exposed.

On the other hand, if the IMD 100 (shown in FIG. 1) determines that the strength of the external magnetic field generated by the MR imaging system has not decreased below the exit threshold, then the strength of the external magnetic field may indicate that the IMD 100 is still within the presence of an external magnetic field generated by an active MR imaging system. As a result, the IMD 100 remains in the MR safe mode of operation.

At 1014, the IMD 100 (shown in FIG. 1) compares the strength of the external magnetic field to an alarm threshold. The alarm threshold is a predetermined external magnetic field strength that represents a very large external magnetic field. For example, the alarm threshold may be 3.0 Tesla, 5.0 Tesla, 8.0 Tesla, 10.0 Tesla, and the like. If the IMD 100 determines that the external magnetic field exceeds the alarm threshold, then the strength of the external magnetic field may indicate it is unsafe for the IMD 100 to remain in the presence of the external magnetic field. The IMD 100 may be incapable of continuing to safely operate when in the presence of the external magnetic field. For example, the IMD 100 may be unable to safely deliver stimulus pulses to the heart 102 (shown in FIG. 1) when exposed to external magnetic fields that exceed the alarm threshold. As a result, flow of the method 1000 proceeds to 1016.

At 1016, the IMD 100 (shown in FIG. 1) switches modes of operation to an alarm mode. In the alarm mode, the IMD 100 may continue to operate in the MR safe mode. For example, the IMD 100 may continue to apply stimulus pulses to the heart 102 while not monitoring cardiac signals of the heart 102. Alternatively, the IMD 100 may change the rate at which the stimulus pulses are applied, and/or may change which chambers of the heart 102 receive the stimulus pulses.

At 1018, the IMD 100 (shown in FIG. 1) activates an alarm to notify the person with the IMD 100 implanted in his or her heart 102 (shown in FIG. 1) of the large external magnetic field. The IMD 100 may activate an internal alarm. For example, the IMD 100 may turn on an alarm located within the IMD 100 that vibrates and/or creates an audible sound for the person to hear. The alarm warns the person to get out of the vicinity of the external magnetic field. Alternatively, the IMD 100 may activate an external alarm by wirelessly transmitting a signal to the external alarm. For example, the IMD 100 may transmit a signal to an external alarm worn by the patient to warn the patient of the large magnetic field.

Flow of the method 1000 proceeds back to 1008 where the IMD 100 (shown in FIG. 1) measures the external magnetic field strength. The IMD 100 may remain in the alarm mode until the IMD 100 determines that the external magnetic field strength has decreased below the alarm threshold. The IMD 100 may sample the external magnetic field until the field measurements decrease below the alarm threshold.

On the other hand, if, at 1014, the IMD 100 (shown in FIG. 1) determines that the field measurements of the external magnetic field do not exceed the alarm threshold, then the field measurements may indicate that the IMD 100 may continue to safely operate in the MR safe mode in the presence of the external magnetic field. Flow of the method 1000 may return to 1006 where the IMD 100 continues to operate in the MR safe mode.

FIG. 11 illustrates a block diagram of exemplary internal components of the IMD 100 in accordance with one embodiment. The IMD 100 includes the housing 110 that includes a left ventricle tip input terminal (V_{L} TIP) 1100, a left atrial ring input terminal (A_{L} RING) 1102, a left atrial coil input terminal (A_{L} COIL) 1104, a right atrial tip input terminal (A_{R} TIP) 1106, a right ventricular ring input terminal (V_{R} RING) 1108, a right ventricular tip input terminal (V_{R} TIP) 1110, an RV coil input terminal 1112 and an SVC coil input terminal 1114. A case input terminal 1116 may be coupled with the housing 110. The input terminals 1100-1114 may be electrically coupled with the electrodes 112-128 (shown in FIG. 1).

The IMD 100 includes a programmable microcontroller 1118, which controls the operation of the IMD 100. The microcontroller 1118 (also referred to herein as a processor, processor module, or unit) typically includes a microprocessor, or equivalent control circuitry, and may be specifically designed for controlling the delivery of stimulation therapy and may further include RAM or ROM memory, logic and timing circuitry, state machine circuitry, and I/O circuitry. The microcontroller 1118 may include one or more modules and processors configured to perform one or more of the operations described above in connection with the method 1000.

A detection module 1120 monitors cardiac signals of the heart 102 (shown in FIG. 1) to identify cardiac events. The detection module 1120 employs one or more algorithms, processes, methods, and analyses to identify cardiac events such as cardiac waveforms, arrhythmias, and the like. The detection module 1120 may measure intervals between cardiac events and/or calculate characteristics of cardiac waveforms to determine when particular cardiac events occur. As described above, the detection module 1120 may automatically switch between normal, MR safe, and alarm modes based on the presence of an external magnetic field.

A control module 1122 identifies a presence of an external magnetic field based on field measurements of the magnetic field. The control module 1122 may receive field measurements from the sensing circuit 804. For example, the control module 1122 may sample the differential voltages generated by the sensing circuit 804 in response to the sensing circuit 804 being located within an external magnetic field. The control module 1122 compares the differential voltages to predetermined thresholds in order to determine whether to switch operating modes of the detection module 1132. In one embodiment, the control module 1122 may calculate a moving average of the samples differential voltages and compare the average to the predetermined thresholds of the MR safe range, the alarm threshold, and/or the exit threshold to determine when the detection module 1132 should switch to the normal operating mode, the MR safe mode, and the alarm mode, as described above.

An alarm module 1136 activates an alarm 1138 in the IMD 100 when the control module 1122 switches the IMD 100 to an alarm mode. For example, the alarm module 1136 may turn on a vibrating or sound-generating alarm 1138 when the control module 1122 determines that an external magnetic field exceeds an alarm threshold, as described above.

The microprocessor 1118 receives signals from the electrodes 112-128 (shown in FIG. 1) via an analog-to-digital (A/D) data acquisition system 1124. The cardiac signals are sensed by the electrodes 112-128 and communicated to the data acquisition system 1124. The cardiac signals are communicated through the input terminals 1100-1114 to an electronically configured switch bank, or switch, 1126 before being received by the data acquisition system 1124. The data acquisition system 1124 converts the raw analog data of the signals obtained by the electrodes 120-138 into digital signals 1128 and communicates the signals 1128 to the microcontroller 1118. A control signal 1130 from the microcontroller 1118 determines when the data acquisition system 1124 acquires signals, stores the signals 1128 in a memory 1134, or transmits data to an external device 1132.

The switch 1126 includes a plurality of switches for connecting the desired electrodes 112-128 (shown in FIG. 1) and input terminals 1100-1114 to the appropriate I/O circuits. The switch 1126 closes and opens switches to provide electrically conductive paths between the circuitry of the IMD 100 and the input terminals 1100-1116 in response to a control signal 1140. An atrial sensing circuit 1142 and a ventricular sensing circuit 1144 may be selectively coupled to the leads 104-108 (shown in FIG. 1) of the IMD 100 through the switch 1126 for detecting the presence of cardiac activity in the chambers of the heart 102 (shown in FIG. 1). The sensing circuits 1142, 1144 may sense the cardiac signals that are analyzed by the microcontroller 1118. Control signals 1146, 1148 from the microcontroller 1118 direct output of the sensing circuits 1142, 1144 to the microcontroller 1118.

An atrial pulse generator 1164 and a ventricular pulse generator 1166 generate pacing stimulation pulses for delivery by the leads 104-108 (shown in FIG. 1) and the electrodes 112-128 (shown in FIG. 1). The atrial and ventricular pulse generators 1164, 1166 may include dedicated, independent pulse generators, multiplexed pulse generators, or shared pulse generators. The pulse generators1164, 1166 are controlled by the microcontroller 1118 via appropriate control signals 1168, 1170 respectively, to trigger or inhibit the stimulation pulses.

In the case where the IMD 100 is intended to operate as an implantable cardioverter/defibrillator (ICD) device, the IMD 100 may detect the occurrence of an arrhythmia, and automatically apply an appropriate electrical shock therapy to the heart aimed at terminating the detected arrhythmia. To this end, the microcontroller 1118 further controls a shocking circuit 1172 by way of a control signal 1174. The shocking circuit 1172 generates shocking pulses of low (up to 0.5 joules), moderate (0.5-10 joules), or high energy (11 to 40 joules), as controlled by the microcontroller 1118. Such shocking pulses are applied to the patient's heart 102 (shown in FIG. 1) through at least two shocking electrodes, such as the coil electrode 120 (shown in FIG. 1), the RV coil electrode 126 (shown in FIG. 1), and/or the SVC coil electrode 128 (shown in FIG. 1).

An impedance measuring circuit 1150 is enabled by the microcontroller 1118 via a control signal 1152. The impedance measuring circuit 1150 may be electrically coupled to the switch 1126 so that an impedance vector between any desired pairs of electrodes 112-128 (shown in FIG. 1) may be obtained. The IMD 100 additionally includes a battery 1154 that provides operating power to the circuits shown within the housing 110, including the microcontroller 1118 and the sensing circuit 804. For example, the battery 1154 may be the power source 802 (shown in FIG. 8) that supplies current to the sensing circuit 804. The IMD 100 includes a physiologic sensor 1156 that may be used to adjust pacing stimulation rate according to the exercise state of the patient.

The memory 1134 may be embodied in a computer-readable storage medium such as a ROM, RAM, flash memory, or other type of memory. The microcontroller 1118 is coupled to the memory 1134 by a suitable data/address bus 1158. The memory 1134 may store programmable operating parameters and thresholds used by the microcontroller 1118, as required, in order to customize the operation of IMD 100 to suit the needs of a particular patient. The operating parameters of the IMD 100 and thresholds may be non-invasively programmed into the memory 1134 through a telemetry circuit 1160 in communication with the external device 1132, such as a trans-telephonic transceiver or a diagnostic system analyzer. The telemetry circuit 1160 is activated by the microcontroller 1118 by a control signal 1162. The telemetry circuit 1160 allows intra-cardiac electrograms, cardiac waveforms of interest, thresholds, status information relating to the operation of IMD 100, and the like, to be sent to the external device 1132 through an established communication link 1164.

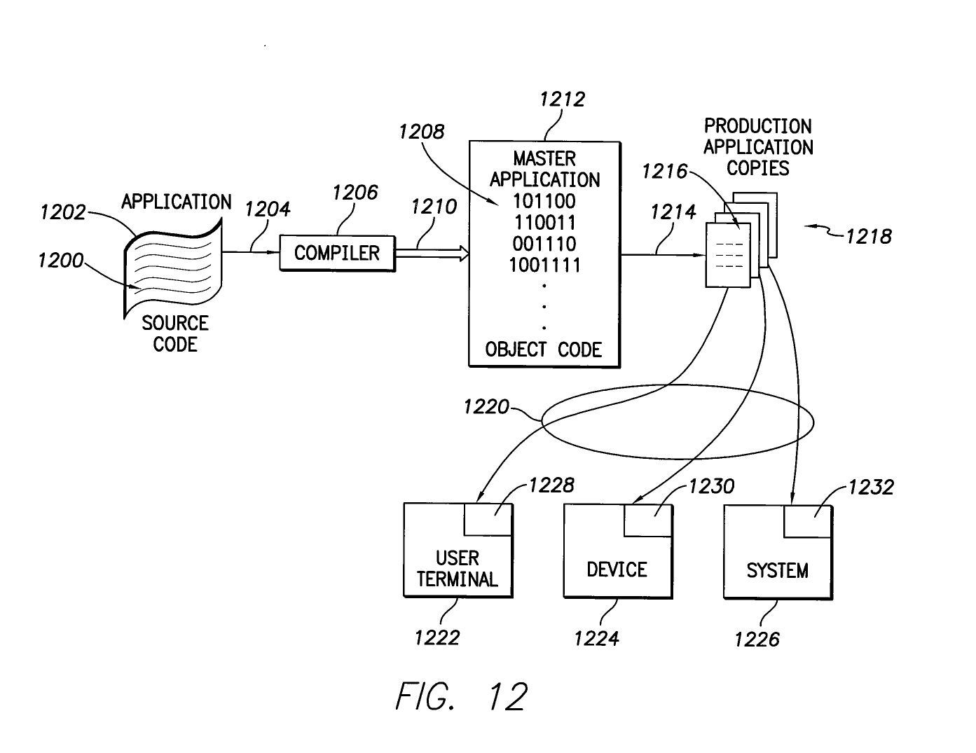

FIG. 12 illustrates a block diagram of example manners in which embodiments of the present invention may be stored, distributed, and installed on a computer-readable medium. In FIG. 12, the “application” represents one or more of the methods and process operations discussed above. The application is initially generated and stored as source code 1200 on a source computer-readable medium 1202. The source code 1200 is then conveyed over path 1204 and processed by a compiler 1206 to produce object code 1208. The object code 1208 is conveyed over path 1210 and saved as one or more application masters on a master computer-readable medium 1212. The object code 1208 is then copied numerous times, as denoted by path 1214, to produce production application copies 1216 that are saved on separate production computer-readable media 1218. The production computer-readable media 1218 are then conveyed, as denoted by path 1220, to various systems, devices, terminals and the like.

A user terminal 1222, a device 1224, and a system 1226 are shown as examples of hardware components, on which the production computer-readable medium 1218 are installed as applications (as denoted by 1228, 1230, 1232). For example, the production computer-readable medium 1218 may be installed on the IMD 100 (shown in FIG. 1) and/or the microcontroller 1118 (shown in FIG. 11). Examples of the source, master, and production computer-readable medium 1202, 1212, and 1218 include, but are not limited to, CDROM, RAM, ROM, Flash memory, RAID drives, memory on a computer system, and the like. Examples of the paths 1204, 1210, 1214, 1220 include, but are not limited to, network paths, the Internet, Bluetooth, GSM, infrared wireless LANs, HIPERLAN, 3G, satellite, and the like.

The operations noted in FIG. 12 may be performed in a widely distributed manner world-wide with only a portion thereof being performed in the United States. For example, the application source code 1200 may be written in the United States and saved on a source computer-readable medium 1202 in the United States, but transported to another country (corresponding to path 1204) before compiling, copying and installation. Alternatively, the application source code 1200 may be written in or outside of the United States, compiled at a compiler 1206 located in the United States and saved on a master computer-readable medium 1212 in the United States, but the object code 1208 transported to another country (corresponding to path 1214) before copying and installation. Alternatively, the application source code 1200 and object code 1208 may be produced in or outside of the United States, but production application copies 1216 produced in or conveyed to the United States (for example, as part of a staging operation) before the production application copies 1216 are installed on user terminals 1222, devices 1224, and/or systems 1226 located in or outside the United States as applications 1228, 1230, 1232.

As used throughout the specification and claims, the phrases “computer-readable medium” and “instructions configured to” shall refer to any one or all of (i) the source computer-readable medium 1202 and source code 1200, (ii) the master computer-readable medium and object code 1208, (iii) the production computer-readable medium 1218 and production application copies 1216 and/or (iv) the applications 1228, 1230, 1232 saved in memory in the terminal 1222, device 1224, and system 1226.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable medical device configured to be exposed to magnetic fields, the device comprising:
a lead including electrodes configured to be positioned within a heart to sense cardiac signals of the heart;
a detection module to monitor the cardiac signals and identify cardiac events based on the cardiac signals;
a field measurement sensor to measure a magnetic field, the sensor to generate a corresponding field measurement based on the magnetic field, the sensor remaining in an unsaturated state when exposed to the magnetic field of at least 0.2 Tesla; and
a control module to identify a presence of the magnetic field based on the field measurement of the sensor and switch operation of the detection module to an MR safe mode based on the field measurement.

2. The implantable medical device of claim 1, wherein the sensor includes alternating layers of paramagnetic material and conductive material.

3. The implantable medical device of claim 2, wherein the layer of the conductive material is disposed between the layers of the paramagnetic material.

4. The implantable medical device of claim 2, wherein the paramagnetic material has a relative magnetic permeability between 1.0 and 1.3.

5. The implantable medical device of claim 2, wherein the paramagnetic material comprises one or more of neodymium, praseodymium, antimony, manganese, beryllium, titanium, zinc, rhenium, niobium, vanadium, tantalum, and tungsten.

6. The implantable medical device of claim 1, wherein the sensor remains in the unsaturated state when the magnetic field is of at least 0.5 Tesla.

7. The implantable medical device of claim 1, wherein the sensor remains in the unsaturated state when the magnetic field is of at least 1.5 Tesla.

8. The implantable medical device of claim 1, wherein the sensor remains in the unsaturated state when the magnetic field is of at least 3.0 Tesla.

9. The implantable medical device of claim 1, wherein the sensor remains in the unsaturated state when the magnetic field is of less than 10.0 Tesla.

10. The implantable medical device of claim 1, wherein the control module switches operation of the detection module to a normal mode when the field measurement of the sensor falls below a predetermined limit, the detection module resuming monitoring of the cardiac signals when the detection module is switched to the normal mode.

11. The implantable medical device of claim 1, further comprising an alarm module to notify a user when the alarm module is in an alarm mode, wherein the control module switches operation of the alarm module to the alarm mode based on the field measurement of the sensor.

12. The implantable medical device of claim 1, wherein the detection module stops monitoring the cardiac signals when switched to the MR safe mode.

13. The implantable medical device of claim 1, further comprising a pulse generator to supply a stimulus pulse through at least one of the electrodes, wherein the control module directs the pulse generator to supply stimulus pulses to the heart when the detection module is switched to the MR safe mode.

14. A method for switching modes of an implantable medical device based on a magnetic field, the method comprising:
sensing cardiac signals originating from a heart over electrodes positioned within the heart;
monitoring the cardiac signals to identify cardiac events;
measuring the magnetic field to which the implantable medical device is exposed to generate a field measurement using a field measurement sensor, the field measurement sensor remaining in an unsaturated state when the magnetic field is of at least 0.2 Tesla;
recognizing the presence of the magnetic field based on the field measurement of the sensor; and
switching operation of the implantable medical device to an MR safe mode based on the field measurement.

15. The method of claim 14, wherein the sensor includes alternating layers of paramagnetic material and conductive material.

16. The method of claim 14, wherein the switching operation comprises changing algorithms used to identify the cardiac events when switched to the MR safe mode.

17. The method of claim 14, further comprising discontinuing the monitoring operation when the implantable medical device is switched to the MR safe mode.

18. The method of claim 14, wherein the switching operation comprises switching the operation of the implantable medical device from the MR safe mode to a normal mode when the field measurement falls below a predetermined limit, further comprising resuming the monitoring operation when the implantable medical device is switched to the normal mode.

19. The method of claim 14, further comprising notifying a user when the field measurement exceeds a predetermined threshold.

20. An implantable medical device comprising:
a lead including electrodes configured to be positioned within a heart to sense cardiac signals of the heart;
a detection module to monitor the cardiac signals to identify cardiac events based on the cardiac signals;
a field measurement sensor to measure a magnetic field to which the implantable medical device is exposed and generate a corresponding field measurement, the sensor comprising alternating layers of paramagnetic material and conductive material; and
a control module to identify a presence of the magnetic field based on the field measurement of the sensor and switch operation of the detection module to an MR safe mode based on the field measurement.

21. The implantable medical device of claim 20, wherein the sensor remains in an unsaturated state when exposed to a magnetic field of at least 0.5 Tesla.

22. The implantable medical device of claim 20, wherein the sensor remains in an unsaturated state when exposed to a magnetic field of at least 1.5 Tesla.

23. The implantable medical device of claim 20, wherein the layer of the conductive material in the sensor is disposed between the layers of the paramagnetic material.

24. The implantable medical device of claim 20, wherein the paramagnetic material of the sensor has a relative magnetic permeability between 1.0 and 1.3.

25. The implantable medical device of claim 20, wherein the paramagnetic material comprises one or more of neodymium, praseodymium, antimony, manganese, beryllium, titanium, zinc, rhenium, niobium, vanadium, tantalum, and tungsten.
